(19) United States
(12) Patent Application Publication
(10) Pub. No.: US 2025/0261665 A1
(43) Pub. Date: Aug. 21, 2025

(54) BOTTOM-UP APPROACH FOR SUSTAINABLE CULTIVATED MEAT PRODUCTION

(71) Applicant: University of Kentucky Research Foundation, Lexington, KY (US)

(72) Inventors: Tyler Barzee, Lexington, KY (US); Ramkumar Tiruvannamalai Annamalai, Buffalo, NY (US)

(21) Appl. No.: 19/054,347

(22) Filed: Feb. 14, 2025

Related U.S. Application Data
(60) Provisional application No. 63/554,630, filed on Feb. 16, 2024.

(51) Int. Cl. A23J 3/26; A23J 3/22; A23L 13/00; A23P 30/25; C12N 5/077

(52) U.S. Cl. CPC A23J 3/26; A23J 3/227; A23L 13/00; A23P 30/25; C12N 5/0658; C12N 2531/00; C12N 2533/54; C12N 2533/74; C12N 2533/90

(57) ABSTRACT

The present disclosure concerns methods and systems for the production of hybrid cell based meat from bio-inks. The bio-inks are cell based to provide layers of protein and connective tissue within the cell based meat. The protein is prepare by culturing muscle cells in a filamentous microcarrier while the connective tissue by culturing pre-adipocytes in polyanionic microcapsules. The bio-inks are prepare by combining the cultured cells with hydrogels.

### CROSS-REFERENCE TO RELATED APPLICATIONS

The present application claims priority to U.S. Provisional Patent Application 63/554,630, filed Feb. 16, 2024, the content of which is hereby incorporated by reference in its entirety.

### BACKGROUND

Global meat production and consumption are rising, driven by population growth, urbanization, and individual income gains. As a result, the demand for meat is estimated to reach 455 million metric tons by 2050. However, intensive meat production required to meet this demand imposes negative externalities on land and water use, public health, and animal welfare. Plant-based meat (PBM) and cell-based meat (CBM) production methods are increasingly considered as alternatives to conventional meat sources. These approaches can eliminate livestock from meat production and increase resource use efficiencies. Although PBM (e.g., tofu) has existed for centuries, new products have recently emerged due to advances in post-harvest processing techniques. However, they are usually more expensive than their animal-based counterparts and it is challenging to retain the nutritional value while achieving desirable mechanical properties. On the other hand, CBM production, also called cultivated meat, is a nascent field that attempts to create meat products from cell cultures rather than whole animals. Thanks to their native organoleptic properties, CBM can precisely emulate meaty flavors, aromas, and textures. Further, alternative food proteins have historically been relegated to “mince” or “ground” meat products due to limitations in food processing technology.

Recent advances in additive biomanufacturing (e.g. 3D printing) have allowed for the creation of the characteristic composite structures of the most valuable meat materials (e.g., steak, sushi filets). Nevertheless, the current state of the art for structured CBM production is utilizing “top-down” approaches, which obtain desired textures by cultivating cells on printed constructs but suffer from substantial mass transfer limitations. As such, a need continues to exist for improved approaches and materials to better mimic meat products and improve consumer appeal.

### SUMMARY

A 1st aspect of the present disclosure, either alone or in combination with any other aspect herein, concerns

### BRIEF DESCRIPTION OF THE DRAWINGS

FIG. 1 shows bottom-up assembly of cell-based meat. FIG. 1A shows adipose tissue constructs. FIG. 1B shows Muscle constructs, and FIG. 1C shows 3D printing of cell-based meat using a carbohydrate bio-ink.

FIG. 2 shows an overview of the overall strategy. FIG. 2A shows polyanionic microcapsules, (Bottom) CS of single capsule showing healthy cell aggregate, FIG. 2B shows fungal pellets, (Bottom) fibrous microarchitecture.

FIG. 3 shows additive Manufacturing. FIG. 3A shows extrusion of hydrogel bio-ink, FIGS. 2B and 2C show stable 3D structures.

### DESCRIPTION

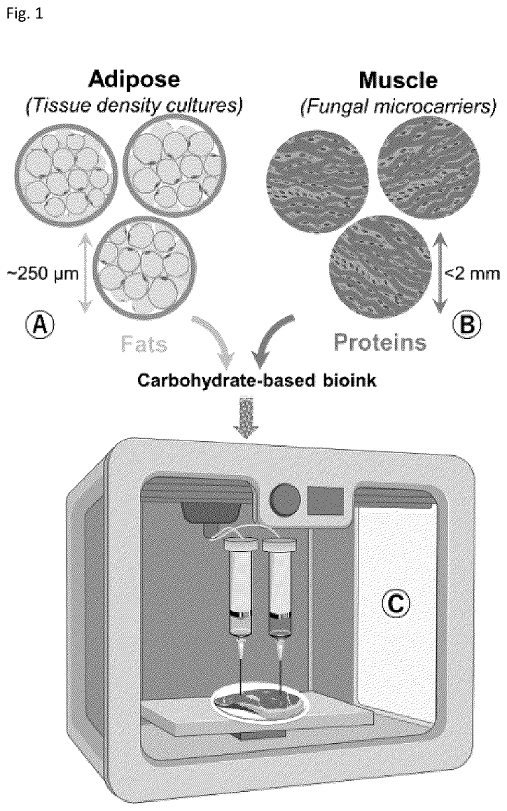

The present disclosure concerns methods and materials for producing or bioprinting meat products or hybrid-CBM. In some aspects, the present disclosure concerns preparing two or more “bio-inks” that can then be used in 3D printing of a hybrid-CBM. In some aspects, each bio-ink can include one or more cell types that can then be printed with other bio-inks to mimic tissue or microtissue. In some aspects, the present disclosure addresses bottlenecks of the top-down approach by providing a “bottom-up” approach, cultivating microtissues before printing a ready-to-consume product (see, FIG. 1).

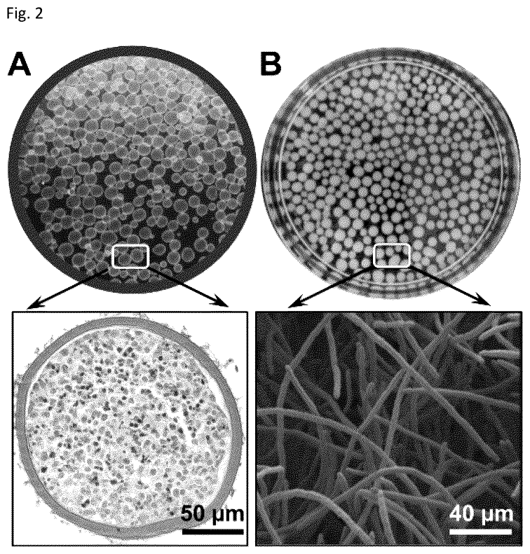

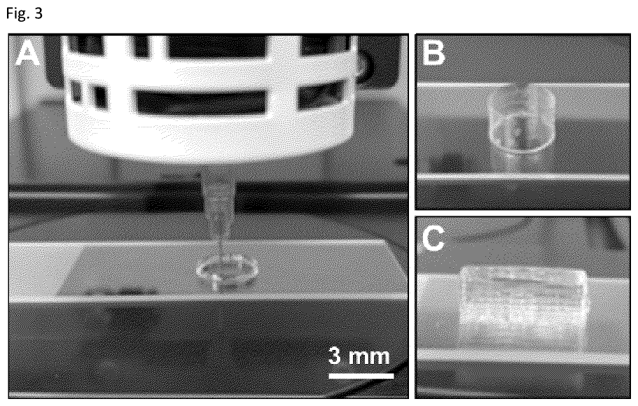

In some aspects, the present disclosure includes three core processes for preparing hybrid-CBM: 1) Creating modular tissue density cultures (see, e.g., FIG. 2A) that enrich adipocyte differentiation and essential fatty acids production in a fat-based bio-ink, 2) Creating modular microcarriers using filamentous fungal pellets (see, e.g., FIG. 2B) to create textured muscles in a muscle based bio-ink, 3) Applying an additive manufacturing approach (see, e.g., FIG. 3) to combine the two modules of bio-inks with hydrogel matrices to create hybrid-CBM.

In some aspects, the present disclose concerns preparing a bio-ink a cultured cells within a structured microenvironment that supports the growth and differentiation thereof. For example, as set forth herein, application of myocytes to a filamentous fungal derived microcarrier allows for muscle cell cultivation. Similarly, adipocyte or pre-adipocyte in a biopolymer allows for fat cell cultivation. The preparation of cultivated cells in a microenvironment wherein they can grow and differentiate therein allows for their harvest and processing into a bio-ink with a hydrogel.

In some aspects, the bio-inks of the present disclosure include cells. The cells can be of one or more type, such as a myocyte, a myoblast, an adipocyte or pre-adipocyte, a fibroblast, a hematopoietic cell, a satellite cell, a stem cell, a progenitor cell, or a combination thereof. As set forth herein, the cells are cultured in microcarriers and/or biopolymers to all for growth and/or differentiation therein. The matured cells can then be applied to a bio-ink by incorporating a hydrogel. The bio-ink can be extruded through an outlet in a 3D printer to assemble a hybrid-CBM. It will be appreciated that the use of two or more different bio-inks allows for the 3D printer to construct hybrid-CBMs that more closely resemble animal flesh. It will also be appreciated that the cells of the bio-inks can be from a particular animal organ to create specialized hybrid-CBMs, such as liver, heart, kidney, lung, thyroid, brain, intestine, and similar. It will also be appreciated that the cells can be derived from any animal, including all vertebrates and invertebrates. In some aspects, the cells are derived from more popular meat sources such as cow, sheep, pig, fish, chicken, and so forth. In some aspects, the bio-inks as set forth herein may further include additional cells such as plant based or plant derived cells.

In some aspects, the present disclosure concerns preparing a protein-based bio-ink, such as a muscle-based bio-ink. In some aspects, muscle cells or myocytes such as skeletal myocytes, cardiac myocytes, and/or smooth myocytes can be used. In some aspects, the present disclosure concerns preparing a lipid-based bio-ink, such as an adipocyte-based bio-ink. In some aspects, adipocytes or pre-adipocyte cells can be used to be cultured to form the bio-ink. It will be appreciated that the bio-ink need not be limited to one particular cell type or a cell type derived from one particular animal or species. For example, a bio-ink may include myocytes and connective tissue, fats, collagens, or other extra-cellular matrix (ECM) materials, as well as other cell types such as adipocytes, fibroblasts, chondrocytes, hematopoietic cells, satellite cells, stem cells, and progenitor cells.

In aspects, the present disclosure concerns preparing a bio-ink by culturing a cell in a carrier, such as a microcarrier, a microcapsule, or a scaffold. Such can include a filamentous fungal scaffold or microcarrier, a polyanionic microcapsule, a biopolymer, decellularized biomass, including plant biomass, bacterial and/or fungal derived fibers, synthetic scaffolds, or similar.

In some aspects, the bio-ink is prepared by adding the desired cell type to a filamentous microcarrier, such as a fungal microcarrier. To create a sustainable protein source, myocytes or myoblasts are contacted, incubated, and grown/differentiated in protein-rich filamentous fungal microcarriers. The inactivated mycelia provides a fibrous scaffold for the growth of myotubes while providing a rich source of vitamins, dietary fiber, essential amino acids, and healthy lipids to the final product. In some aspects, the bio-ink is prepared by adding the desired cell type to a biopolymer. As set forth herein, adipocytes or pre-adipocytes can utilize a biopolymer to grow and mature over time, which then can be coupled into a hydrogel and used as a bio-ink to provide a fat portion of a hybrid-CBM.

It will be appreciated that the density of the microcarrier or polyanionic microcapsule with a biopolymer and the number/volume of cells will affect the resulting quality of the bio-ink. The number of cells can be controlled through cell culturing techniques understood in the field. The microcarriers are similarly established materials. Typically, cells can be cultured for a period of time ranging from about 2 to 15 days, including about 3, 4, 5, 6, 7, 8, 9, 10, 11, 12, 13, and 14 days. Spherical fungal microcarriers are fabricated by isolating conidia solution from a mature plate, inoculating and agitating for a set period of time. The resulting microcarriers are isolated, heat-deactivated and/or chemically deactivated and then seeded with the desired cells, e.g. myocytes, maintained in growth media and then optionally switched to a differentiation media.

In some aspects, the microcarriers with cells therein can then be mixed with a hydrogel such as gelatin-alginate, corn starch, potato starch, pectin, cellulose, chitosan, collagne, hyaluronic acid, guar gum, agarose, dextran, or similar to achieve a bio-ink of desired rheology and density, such that the bio-ink can flow or be pumped through a desired outlet, such as a nozzle or a gauged needle and printed. In some aspects, the hydrogel is of a food-grade and is of sufficient shear-thinning nature that it can be deployed in a 3D printer. In addition to varying the microcarrier density and cell number/volume therein, the rheology of the bio-ink can be varied by the volume and/or density of hydrogel utilized therewith. It will be apparent that the diameter of the outlet used for extrusion/printing the bio-ink can determine the needed rheology of the bio-ink. If the bio-ink does not flow sufficiently through the outlet, the bio-ink will not effectively print or extrude.

In some aspects, prior to suspension with a hydrogel, the microcarriers with cells therein can be further processed prior to reconstitution with a hydrogel. In some aspects, the microcarriers can be dried, freeze-dried or otherwise processed and/or preserved for storage and/or to halt cell growth therein. It will be appreciated that contact with a hydrogel will reconstitute and/or rehydrate the cells therein. Dried or freeze dried microcarriers can be further processed to achieve a uniform size, such as through milling and or sieving or other sorting methods known in the art.

It will also be appreciated that a bio-ink may be further treated and/or processed prior to extrusion in a printing process. For example, a bio-ink may receive one or more dyes to achieve a desired color. Further, a bio-ink may receive addition nutrients such as vitamins, minerals, calories, proteins, amino acids, fats, carbohydrates, or other items of nutritional value.

In some aspects, the present disclosure concerns preparing a second bio-ink. In some aspects, the second bio-ink is a lipid-based bio-ink. In some aspects, the second bio-ink is a connective tissue-based bio-ink. Fats substantially contribute to the taste, aroma, tenderness, and high-energy content of meat. To create robust fat tissues, a 3D culture system with minimal mass transport limitation is necessary. Pre-adipocytes can be encapsulated and differentiated by providing a suspension of cells in a polyanionic solution such as carboxymethylcelluloses (CMC)) and dispensing droplets thereof into a polyanionic microcapsule with a biopolymer such as a cationic biopolymer, including chitosan, cationic gelatin, cationic dextran, cationic cellulose, and cationic cyclodextrin. Capsule membranes form instantaneously by ionic complexation between oppositely charged polymers. Fully matured capsules can then be washed and cultured in growth media for a desired period of time. The second bio-ink can then be either directly mixed with a hydrogel or optionally dried or freeze dried first and then reconstituted as a hydrogel. The second bio-ink may similarly receive one or more dyes to achieve a desired color. Further, the second also bio-ink may receive addition nutrients such as vitamins, minerals, calories, proteins, amino acids, fats, carbohydrates, or other items of nutritional value.

In some aspects, the biopolymers with cells therein can then be mixed with a hydrogel such as gelatin-alginate to achieve a bio-ink of desired rheology and density, such that the bio-ink can flow or be pumped through a desired outlet, such as a nozzle or a gauged needle and printed. In addition to varying the biopolymer density and cell number/volume therein, the rheology of the bio-ink can be varied by the volume and/or density of hydrogel utilized therewith. It will be apparent that the diameter of the outlet used for extrusion/printing the bio-ink can determine the needed rheology of the bio-ink. If the bio-ink does not flow sufficiently through the outlet, the bio-ink will not effectively print or extrude.

In some aspects, a third bio-ink or even more can be prepared. Such can include culturing of a desired cell type with a support, such as a biopolymer or a filamentous microcarrier as set forth herein. Such may also include dyes and/or addition of further nutritional materials as the bio-ink is prepared with a hydrogel prior to extrusion/printing.

In some aspects, the present disclosure concerns bioprinting using the bio-inks as set forth herein. Each microtissue module can be harvested, colored with pigments to resemble fat and adipose tissue, and homogenized within a hydrogel such as gelatin-alginate to form each bio-ink. Each bio-ink can then be extruded through separate nozzles with a 3D bioprinter. For example, fat and protein bio-inks can be deposited in desired patterns to create marbled CBM that is ready to package and cook without additional cultivation steps that are required for top-down approaches.

The present disclosure utilizes bottom-up bioprinting to fabricate a CBM. In contrast to top-down approaches to creating structured “marbled” cultivated meat, the present disclosure cultivates cells on 3D-scaffolds that provide the environment and support to deliver desired characteristics to the final product while maximizing efficiency of cultivation. The fungal-derived microcarriers are structurally similar to muscle fibers and the polyanionic microcapsules resemble adipose tissue. The present disclosure cultivates cells in optimal conditions and avoids detrimental mass transfer limitations that plague top-down approaches and result in costly cultivation and assembly. The present disclosure allows direct printing of a final marbled product without the need for additional cultivation steps. It is a further aspect that the delivery of structural characteristics with these specific carriers can also provide significant reduction in differentiation media costs.

The present disclosure therefore has application in food technology. Cultivated meat and alternative proteins in general are a rapidly expanding area of commercial interest and investment. The United States FDA approved the first cultivated meat product in 2023 from Upside Foods whose first product is cultivated chicken nuggets. The main limitations in the industry are high costs associated with cultivation and harvest, especially for low value, high throughput unstructured products like chicken nuggets. The present disclosure has applications in much higher value markets for structured products such as steak and fish, pork, and chicken filets. Additionally, this technology could be extended to other non-meat materials for extremely nascent areas of cellular agriculture and additive bio-manufacturing such as printed fruits and vegetables and other animal product analogues (delicacy organs, foie gras, roe, etc.).

### EXAMPLES

The central working methods of the present disclosure are that 1) Modular tissue-density constructs can evade mass-diffusion limitations to create bulk tissues, and 2) Combining adipose, muscle, and filamentous fungi microtissues in a hydrogel matrix creates hybrid-CBM with composition and texture comparable to animal meat (FIG. 1).

Fabricate extrudable adipose microtissues that eliminate diffusion limitations while maintaining issue density. Pre-adipocytes are encapsulated in chitosan-carboxymethyl cellulose microcapsules (˜250 μm); allowed to proliferate and differentiate therein. An endpoint includes microtissues with ˜80% fat by dry mass. Fats substantially contribute to the taste, aroma, tenderness, and high-energy content of meat. To create robust fat tissues, a 3D culture system with minimal mass transport limitation is necessary. A modular microcapsule system was previously developed that can maintain highly metabolic cells at tissue densities (˜6×107 cells/cm3) with no signs of central necrosis (Annamalai, PloS one 9, e84287 (2014)). The aim is to encapsulate and differentiate pre-adipocytes as described. Microencapsulation: 5 million 3T3-L1pre-adipocytes will be suspended in 1 ml of a polyanionic soln. (1.5 wt % carboxymethylcelluloses (CMC)), droplets (˜200 μm) will be generated using a 24 gauge Teflon catheter and dispensed into 30 ml of stirred chitosan (1.2 wt %). Capsule membranes will form instantaneously by ionic complexation between oppositely charged polymers. Fully matured capsules will be washed, cultured in growth media for 4 weeks, and characterized for viability (CalceinAM LIVE/DEAD assay), proliferation (PicoGreen Assay), adipogenic differentiation (Oil-Red-O Stain), and lipid content (sulfo-phospho-vanillin method).

Extrudable muscle constructs are created by seeding myoblasts on inactivated filamentous fungal microcarriers. Filamentous fungus Aspergillus oryzae are cultured to produce fibrous microcarriers (˜1 mm); myoblasts are then cultured on the surface of fungal microcarriers and allowed to proliferate and differentiate thereon, as well as deposit/establish ECM. An endpoint includes creating constructs with 50% protein by dry mass. To create a sustainable protein source, we propose growing muscle cells in protein-rich filamentous fungal microcarriers. The inactivated mycelia will provide a fibrous scaffold for the growth of myotubes while providing a rich source of vitamins, dietary fiber, essential amino acids, and healthy lipids to the final product. Microcarriers: Spherical fungal microcarriers will be fabricated by isolating conidia solution from a mature plate, inoculating in potato dextrose broth (PDB), and agitating at 150 rpm for 2 days. The resulting microcarriers will be isolated, heat-deactivated, seeded with myoblasts (C2C12 cell line), maintained in growth media (DMEM+10% FBS), and switched to differentiation media (DMEM+2% HS). Myotube formation and muscle maturation will be characterized using SEM, IHC, IF (Myosin HC), and qPCR (Myog, Myod1, Actn, Ppia).

3D hydrogel bio-ink is formed by extruding adipose and muscle microtissues and forming marbled hybrid-CBM. A shear-thinning alginate and gelatin-based hydrogel composite is formed; fat and muscle microtissues in bio-ink formulations are individually suspended therein to 3D print bulk fat and muscle tissues; marbled hybrid-CBM is printed. An endpoint includes a CBM with 30% protein, 40% fat, and 30% carbohydrates and a texture comparable to steak. Microtissue modules are harvested and homogenized in gelatin-alginate bio-ink and validated for printability and stability, reproducibility, and mechanical properties. 3D Printing: An Allevi 3TM tri-head 3D bioprinter to study the effect of printing parameters (e.g., nozzle diameter, pressure) on printability and the quality of the resulting prints. Bio-ink printability will be quantified according to published methods to obtain values of spreading ratio and filament collapse area with nozzle diameters of 0.26 mm and 0.51 mm (25 and 21 Gauge, respectively). In addition, the texture profile of constructs will be evaluated by uniaxial compression testing (Instron).

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The drawings are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. A method for preparing hybrid cell based meat (CBM) comprising:
preparing a protein bio-ink;
preparing a lipid bio-ink; and
extruding the protein bio-ink and the lipid bio-ink on a surface to provide a marbled hybrid-CBM.

2. The method of claim 1, wherein the protein bio-ink is prepared by culturing myocytes or myoblasts or a combination thereof in a carrier

3. The method of claim 2, wherein the carrier is a filamentous fungal microcarrier.

4. The method of claim 3, further comprising contacting the filamentous fungal microcarriers with cells cultured therein with a hydrogel.

5. The method of claim 3, wherein the hydrogel comprises gelatin-alginate.

6. The method of claim 3, wherein the filamentous fungal microcarriers with cells cultured therein are freeze-dried prior to contact with the hydrogel.

7. The method of claim 1, wherein the lipid bio-ink is prepared by culturing pre-adipocytes in a carrier.

8. The method of claim 7, wherein the carrier is a polyanionic microcapsule with a biopolymer.

9. The method of claim 8, wherein the biopolymer is a cationic biopolymer.

10. The method of claim 9, wherein the cationic biopolymer is selected from chitosan, cationic gelatin, cationic dextran, cationic cellulose, and cationic cyclodextrin.

11. The method of claim 8, further comprising contacting the polyanionic microcapsule with the biopolymer with pre-adipocyte cultured therein with a hydrogel.

12. The method of claim 11, wherein the hydrogel comprises gelatin-alginate.

13. The method of claim 11, wherein the polyanionic microcapsule with the biopolymer with pre-adipocyte cultured therein are freeze-dried prior to contact with the hydrogel.

14. The method of claim 1, wherein the protein bio-ink and/or the lipid bio-ink are dyed.

15. The method of claim 1, wherein the protein bio-ink and/or the lipid bio-ink further comprise a carbohydrate, a mineral, a vitamin, an amino acid, a peptide, or a combination thereof.

16. The method of claim 1, further comprising extruding the protein bio-ink to provide muscle constructs within the hybrid-CBM.

17. The method of claim 1, further comprising extruding the lipid bio-ink to provide connective microtissue within the hybrid-CBM.

18. A system for producing CBM comprising a 3D bio-printer operably connected to a protein bio-ink and a lipid bio-ink.

19. The system of claim 18, wherein the protein bio-ink comprises myoblasts and a carrier.

20. The system of claim 18, wherein the lipid bio-ink comprises pre-adipocytes and a carrier.
